(19) United States
(12) Patent Application Publication
(10) Pub. No.: US 2008/0146887 A1
(43) Pub. Date: Jun. 19, 2008

(54) Intelligent personal health management appliances for external and internal visualization of the human anatomy and for dental/personal hygiene

(76) Inventors: Raman K. Rao, Palo Alto, CA (US); Sunil K. Rao, Palo Alto, CA (US); Rekha K. Rao, Palo Alto, CA (US)

Correspondence Address: COURTNEY STANIFORD & GREGORY LLP, P.O. BOX 9686, SAN JOSE, CA, 95157, US

(21) Appl. No.: 11/079,917

(22) Filed: Mar. 13, 2005

Related U.S. Application Data
(63) Continuation-in-part of application No. 11/001,668, filed on Nov. 30, 2004.

(51) Int. Cl. A61B 5/00

(52) U.S. Cl. 600/300

(57) ABSTRACT

A system, method and apparatus for real time visualization of various internal and external parts of the human anatomy, using an intelligent visualization appliance that operates with wired or wireless communication capabilities for communication with one or more other intelligent personal health management appliances, cellular telephone, PDA, lap top computers and other mobile devices, stationary devices and local, central and Internet servers. The intelligent visualization appliance is additionally capable of cleaning various parts of the human body and application of medications. Further including an intelligent dental hygiene system and apparatus for visualization and customized cleaning in conjunction with an intelligent dental hygiene appliance. The intelligent visualization appliance and the intelligent dental hygiene appliance is enabled with internal processing, storage, software, display and other capabilities for standalone operation or operation in conjunction with a mobile device, stationary device and one or more servers in a wired or wireless network.

### CROSS REFERENCE TO RELATED APPLICATIONS

The present application is a continuation-in-part of co-pending application Ser. No. 11/001,668 filed on Nov. 30, 2004 titled: Real time personal health management using mobile intelligent appliances and gastronomic information.

### ABBREVIATIONS

### Cellular Telephone is abbreviated as CT

### Mobile Device is abbreviated as MD

### Intelligent Personal Health Management Appliance is abbreviated as IPHMA

### Personal Health Management is abbreviated as PHM

### The terms Appliance and Device are used interchangeably.

### Intelligent Visualization Appliance is abbreviated as IVA

### Intelligent Dental Hygiene Appliance is abbreviated as IDHA

### BACKGROUND OF THE INVENTION

There is a need for a new class of intelligent appliances, referred in here as intelligent personal health management appliances, that enable the individual to personally acquire one or more types of visual information in real time, said information being related to the internal cavities of the individual or related to the external parts of the human anatomy including the skin and body profile of the individual; archive said information for correlation with factors such as diet and other activities including physical exercise/mental activity/stress and other factors, for personal health management by the individual him/her self or for personal health management in conjunction with a qualified health professional. The present invention teaches novel methods and novel appliances for real time data acquisition of visual biological information, for archiving said data for contemporaneous utility or for utility at a selected time, for analyzing said data, for determining trends, for communication of said visual information data/trends to one or more health professionals and for comprehensive real time management of the personal health of an individual in a cost effective manner in accordance with the quality/cost objectives acceptable to the individual.

### SUMMARY OF THE INVENTION

The present invention enables mobile devices such as cellular telephones, PDAs, laptop computers and other devices to perform the additional functions of acquiring and processing visual information related to the internal cavities of the human body and the external parts of the human body including the environment in which the person is present. The acquisition of the visual information is performed directly by cellular telephones, PDA's, laptop computers and other devices utilizing a built-in image acquisition capability such as in conjunction with a built-in camera feature and or alternately in conjunction with external attachments that facilitate different types of image acquisition.

In addition a new class of novel intelligent appliances and devices which are specifically designed for the acquisition and processing of visual information related to the internal cavities of the human body and external parts of the human anatomy including the environment that the individual is present in are disclosed. These intelligent appliances are configured with image acquisition and image processing capabilities and additionally may include command, control, compute and wired or wireless communication capabilities with a central server and or a mobile device/stationary device such as a personal computer, cellular telephone, PDA, lap top computer and other devices.

The present invention teaches novel methods and appliances to diagnose, communicate and solve various personal health management problems not addressed in the prior art including the ability to visualize various internal and external human anatomical features. The individual user is enabled via these intelligent visualization appliances to observe, track, and correlate various health problems with various other related factors such as diet to better understand the health problem and to better treat the health problem by him/her self or in conjunction with a trained health care professional.

Various important objectives of the present invention are enumerated in detail below. Other objectives may become apparent to those knowledgeable in the art and the invention is not to be construed as limited to the specific objectives enumerated herein:
1. An objective of the present invention is an intelligent personal health management appliance for visual examination [for self examination and or examination by another individual including a trained health care professional] of the internal cavities of a human body such as the mouth, nose, throat, ears, rectum, vagina and other anatomical parts of the human body. The intelligent personal health management appliance is normally intended to have an internal camera or other image acquisition capabilities and or enabled for operation with external image acquisition devices/appliances such as a camera enabled communication and computation device. The intelligent visualization appliance is intended to be operable with wired or wireless communication capabilities. This intelligent personal health management appliance is referred to as an intelligent visualization appliance.
2. An objective of the present invention is to enable the intelligent visualization appliance to be uniquely identifiable by one or more means including a unique IP [Internet Protocol] address, a RF ID tag [Radio Frequency Identification] and other identification means.
3. Another objective of the present invention is to enable an intelligent visualization appliance to be uniquely co-relatable to a specific individual for dedicated use by that individual in conjunction with a user identification method and an intelligent visualization appliance identification system, including an RF ID Tag that is attached or imbedded within the intelligent visualization appliance.
4. Another objective of the present invention is to enable one single intelligent visualization appliance to be used by more than one individual with appropriate selection of user identification.
5. Another objective of the present invention is to enable the intelligent visualization appliance to be used in conjunction with the RF ID Tag of a specific individual and the RF ID Tag system of the intelligent visualization appliance and or other types of people and appliance identification systems.
6. Another objective of the present invention is to enable a single intelligent visualization appliance to be used for multiple purposes with appropriate cleaning and sanitizing procedures.
7. Another objective of the present invention is to enable a single intelligent visualization appliance to be used for a plurality of uses in conjunction with appropriate sanitary and disposable sleeves [including transparent sleeves], attachments and other safety and sterilization procedures.
8. Another objective of the present invention is to enable a single intelligent visualization appliance to be used by a plurality of individuals in conjunction with appropriate sanitary and disposable sleeves, attachments and other procedures.
9. Another objective of the present invention is to enable the design and configuration of an intelligent visualization appliance, for a specific application, such as exclusively for the nostrils, for ears, for mouth and other human anatomical cavities.
10. Another objective of the present invention is to enable an intelligent visualization appliance for use external of the human body such as for visualization of the skin, front torso, back and other parts of the body.
11. Another objective of the present invention is to enable the intelligent visualization appliance with image acquisition, image processing, and image storage and image communication capabilities; and further including voice and data communication capabilities.
12. Another objective of the intelligent visualization appliance is wired or wireless communication with a plurality of other devices, that include cellular telephones, personal digital assistants PDAs, laptop computers, personal computers, servers [including a central server, a local server, a network server and an escrow server], other intelligent personal health management appliances and other stationary and mobile devices.
13. Another objective of the present invention is the secure acquisition, secure processing, secure storage and secure transmittal of personal images [including still images and video and other voice and data information] within the intelligent visualization appliance itself, within/in conjunction with a mobile device, a stationary device, a server or a selected combination thereof.
14. Another objective of the present invention is for the intelligent visualization appliance to used in conjunction with a home network including a home health care network, a health care professional network such as in a doctors office or a hospital and other environments.
15. Another objective of the present invention is to enable comparison of visual information from first time period to a second time period for determination, evaluation and treatment of various conditions that manifest themselves visually; by the individual him/her self including in conjunction with a trained health care professional to who the visual and other information is provided proximately or remotely by wired or wireless communication methods.
16. Another objective of the present invention is to enable the use of an intelligent visualization appliance to be used independently or in conjunction with a mobile device such as a cellular telephone, PDA, laptop computer and other mobile and stationary devices for use by the individual while on the move or in a fixed location.
17. Another objective of the present invention is to enable the personal visualization information to be either communicated by wired or wireless means or physically taken to the doctor's office or always available with the individual for emergency and other uses.
18. Another objective of the present invention is to utilize a stationary or mobile communication device with a built-in camera/image acquisition capability for intelligent visualization external of the body such as the skin and other extremities; and internal to the human body visualization in conjunction with special intelligent visualization appliances/attachments appropriate for the intended application.
19. Another objective of the present invention is to enable the co-relation of visualization information with other factors such as physical activity, exercise, sports and other stressful activities including bug bites.
20. Another objective of the present invention is to enable the co-relation of visualization information to the ingestion of different types of foods, prescription medications and other material ingested.
21. Another objective of the present invention is to enable the tracking of visualized information over a time period to determine the size of a mole or other skin lesions and other 5v conditions for further communication of the visualized information for objective analysis by a trained health care professional.
22. Another objective of the present invention is enable the posture, the stomach shape and other anatomical visual features to be acquired, processed, stored and communicated internal of the intelligent visualization appliance and external of the intelligent visualization appliance; and further co-related with diet and exercise.
23. Another objective of the present invention is an intelligent personal hygiene appliances, that enable the visualization of the internal cavities of the human body/external extremities of the human body and further enable the cleaning and visualization of the selected body cavity or body location contemporaneously, iteratively or at a selected time and in selected sequence using water and one or more types of other cleaning fluids that include gases. The intelligent visualization appliance is enabled with a fixed camera/image acquisition capabilities [including a plurality of cameras] or flexible means that enable bending of the image head or other parts of the intelligent appliance.
24. Another objective of the present invention is an intelligent mirror system that includes a display either imbedded [or other wise included] that displays various selected visualization and other information provided by an intelligent visualization appliance, a mobile device, a stationary device and a server by wired or wireless communication means.
25. Another objective of the present invention is an intelligent dental hygiene appliance/intelligent tooth brush that provides visual images before initiation of teeth cleaning, during teeth cleaning, after teeth cleaning; and further provides customized teeth cleaning per selected and personalized programs including relating the teeth status to the different types of foods ingested and the frequency of teeth cleaning.
26. Another objective of the present invention is to enable each intelligent dental hygiene appliance with an appliance ID such by means of an RF ID Tag to uniquely identify each intelligent dental hygiene appliance to a specific individual; further tracking the teeth cleaning activities [or the absence thereof] of each adult/child in a database.
27. Another objective of the present invention is to enable the intelligent dental hygiene appliance to be configured for delivery of different types of tooth paste [and cleaning fluids] via various cartridges or other means including customized flavors or pre programmed flavors.
28. Another objective of the present invention is to store appropriate visual information and other information in one or more secure databases such as in an escrow server for secure access by authorized individuals/entities.
29. Another objective of the present invention is to enable storage of visual information including X-Ray images, MRI and other images within the intelligent visualization appliance or a mobile device such as a cellular telephone for ready and emergency access by the individual himself and or by authorized individuals/entities.
30. Another objective of the present invention is to enable the intelligent visualization appliance/the intelligent dental hygiene appliance to be configured with wired or wireless communication capabilities for communicating with a central server, peripheral devices and other intelligent appliances in network architecture. The intelligent visualization/dental hygiene appliance being enabled with electronics, processing and internal storage capabilities and being able to operate in a stand alone manner or in conjunction with the capabilities of a central server, a mobile communication/computational device or a combination thereof.
31. Another objective of the invention is to enable personal health management in conjunction with health care providers such as doctors and nurses and institutions such as hospitals for cost effective, remote and real time acquisition of visualization information and measured/monitored information such as heart rate, temperature and other data acquired and provided by different types of personal health management appliances; and other related information such as the foods ingested, the medications ingested, exercise and other information.
32. Another objective of the present invention is to enable personal databases and the maintenance of selected and authorized information related to the individual in the databases of health care providers and institutions for the health management of individuals by the individual him/her self or in conjunction with a health care provider.
33. Another objective of the present invention is to enable a secure escrow server to maintain and provide selected personal visualization information to authorized individuals at the selected time for personal health management

Other objects, features and advantages of the present invention will become apparent from the following detailed description when taken in conjunction with the accompanying drawings.

### BRIEF DESCRIPTION OF THE EMBODIMENTS OF THE INVENTION

The accompanying drawings, which are incorporated in and form a part of this specification, illustrate embodiments of the invention and, together with the description, serve to explain the principles of the invention:

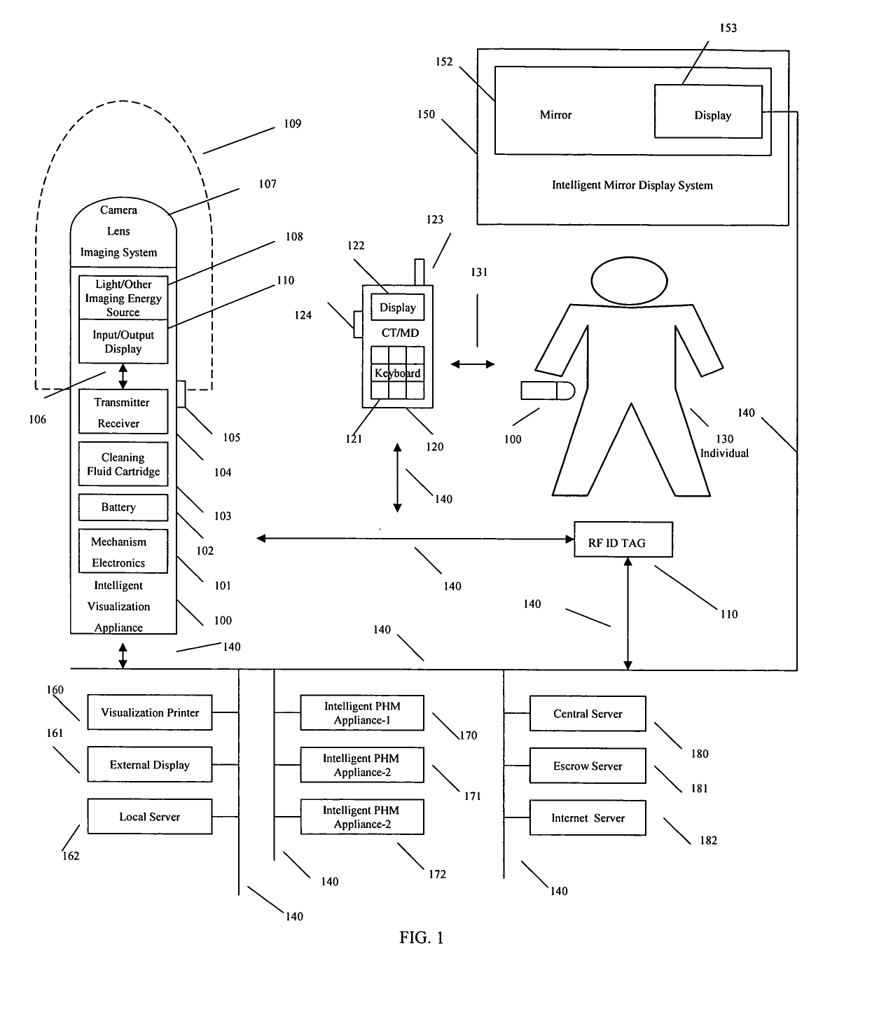

FIG. 1 shows an intelligent visualization appliance for internal/external visualization of the human body, a RF ID Tag identifying the user and a mobile device associated with a user in a wired or wireless networking environment with a central server and other servers. The intelligent internal visualization appliance is shown with an optional cleaning capability and a cleaning fluid reservoir/cartridge[s]. The figure shows one camera/image acquisition head, however multiple cameras/image acquisition heads are intended by reference. The image acquisition may be by optical or other means including X-Ray, Ultra sound and other methods. The figure shows a rigid structure for illustrative ease, however various parts of the intelligent appliance are intended to be flexible and capable of rotation and other manipulations including one or more types of mechanical, ultrasound and other movements. While the intelligent appliance is illustrated with battery power, other types of power sources such as line power, solar power and or a power source from another device via an attachment is intended by reference. The intelligent appliance is intended for operation by wired or wireless communication means of voice, data, images, audio, video and other means of communication utilizing one or more types of communication protocols.

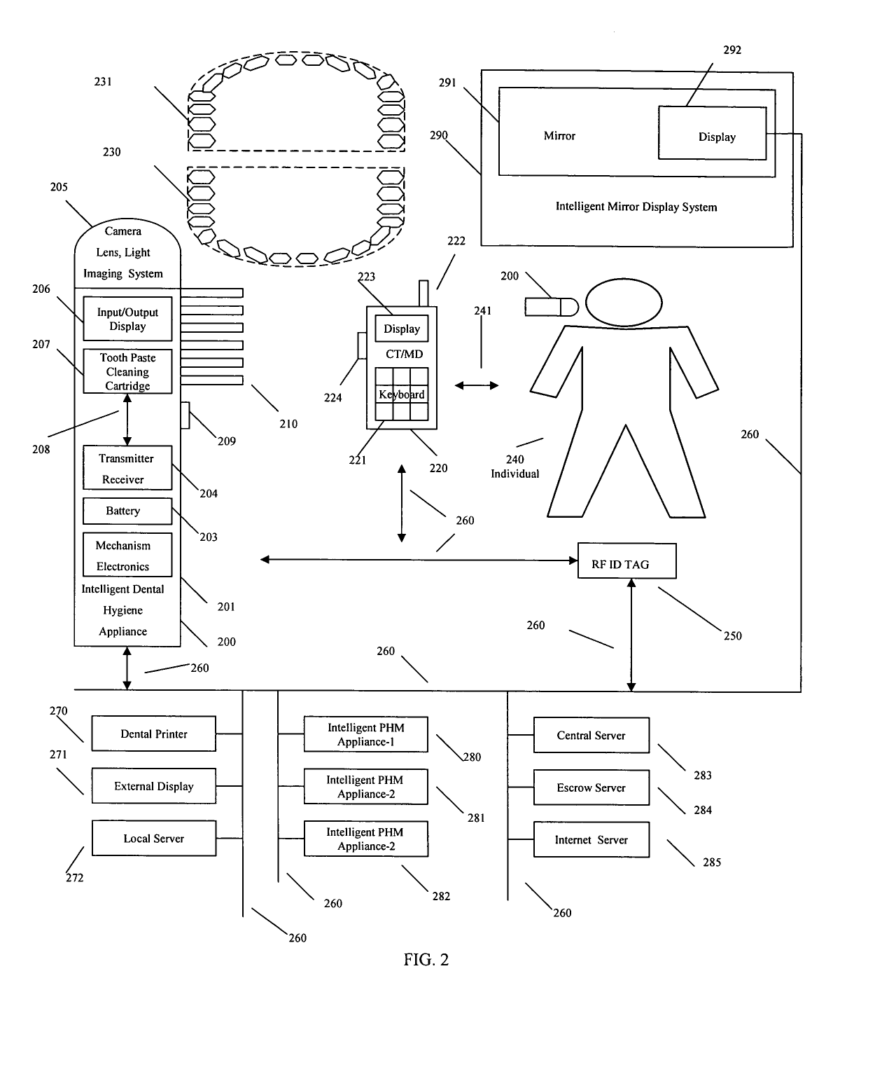

FIG. 2 shows an intelligent dental hygiene appliance for visualization of the mouth/teeth and for brushing/cleaning, a RF ID Tag identifying the user and a mobile device associated with a user in a wired or wireless networking environment with a central server and other servers. The figure shows one camera/image acquisition head, however multiple cameras/image acquisition heads are intended by reference. The figure shows a rigid structure for illustrative ease, however various parts of the intelligent appliance are intended to be flexible and capable of rotation and other manipulations including one or more types of mechanical, ultrasound and other movements. While the intelligent appliance is illustrated with battery power, other types of power sources such as line power, solar power and or a power source from another device via an attachment is intended by reference. The intelligent appliance is intended for operation by wired or wireless communication means of voice, data, images, audio, video and other means of communication utilizing one or more types of communication protocols.

FIG. 3 shows a table that enables personal visualization information to be tracked and recorded for use by the individual and or by health care professionals.

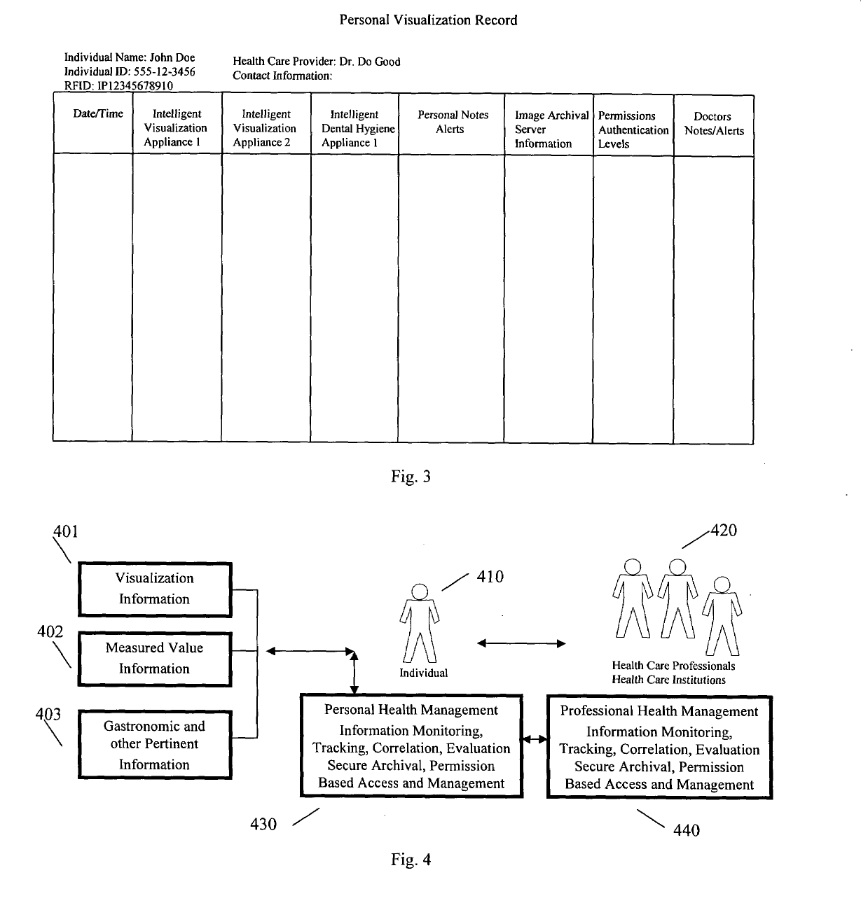

FIG. 4 shows the system that enables the visualization information, various measured information and other pertinent information is used for personal health management by the individual him/her self or in conjunction with health care professionals.

### DETAILED DESCRIPTION OF THE PREFERRED EMBODIMENTS

Reference will now be made in detail to the preferred embodiments of the invention, examples of which are illustrated in the accompanying drawings. While the invention will be described in conjunction with the preferred embodiments, it will be understood that they are not intended to limit the invention to those embodiments. On the contrary, the invention is intended to cover alternatives, modifications and equivalents, which may be included within the spirit and scope of the invention as defined by the appended claims.

For example, the embodiments that follow relate to a networking architecture, a system and apparatus for enabling personal visualization/health management in conjunction with different intelligent appliances that are mobile and or wireless, but are intended to include stationary units as well, such as personal computers (PCs) and stationary intelligent and non intelligent appliances; wherein as an illustration a non intelligent appliance is enabled with an external wireless capability and external networking capability by means of attachments and other methods.

Further, the personal visualization/dental hygiene/health management inquiry or request can be in any form of intelligence, such as key entries from a keyboard, voice in any language, graphics such as a key click on a graphic page, mouse clicks on a view, or even tactile responses or depressions of a foot pedal. Required translations, such as from one language to another or from a tactile entry to a voice command, are made automatically.

As another example, personal visualization/dental hygiene/health management is enabled in conjunction with one or more types of information including gastronomic information, one or more personal and/or common databases, storage, processors and software resident on one or more devices/servers located within the device itself, a local server, a central server, a network server or located across a network including the Internet. The architecture system and apparatus may have other applications, for example in a hospital/institutional/professional environment, and the embodiments described herein are for illustrative purposes and are not to be construed as limiting the present invention.

The present invention is a personal visualization/dental hygiene/health management system comprising of various types of intelligent personal visualization/health management appliances that are designed and used for specific personal visualization/health management applications. The intelligent personal visualization/health management appliances are used in a standalone manner or in conjunction with various gastronomic inquiry and information systems, since the health of an individual including the visual manifestations of the individual's health generally pivots around the ingestion of food, ingestion of other substances, physical activities and mental activities.

The intelligent personal visualization/dental hygiene/health management appliance is configured for the real time acquisition of various data related to the individual, storing the acquired data within said appliance and or on a central server, displaying the selected information on said device or another display device, making an information inquiry including health factors and gastronomic factors to an information server having a personal health and gastronomic database. The present invention includes displaying [image and data] a response to the inquiry related to personal health and gastronomic information from the information server/central server on the selected display device including the intelligent personal visualization/dental hygiene/health management appliance.

The present invention also includes sending a personal health and or a personal visualization inquiry from the intelligent personal health management appliance/display device to the information server, preparing the selected personal health/personal visualization response to said inquiry on the information server, and receiving the response to the inquiry from the information server with an intelligent personal visualization/health management appliance and other devices that include a cellular telephone/a mobile device.

A preferred embodiment includes formatting the personal visualization/health/personal visualization response to the selected inquiry on the display device, efficiently searching for the relevant visualization information, efficiently accessing and acquiring one or more visualization information and displaying the formatted personal visualization/health response to the inquiry on the display device and storing/retrieving said information on one or more databases located on the intelligent appliance itself, on a mobile device such as a cellular telephone, personal digital assistant, lap top computer, other mobile devices, stationary devices and one or more servers such a local server, a central server, a network server and a escrow server.

The escrow server and or other servers securely stores personal visualization/health and other information and provides access to the selected information to selected/permitted individuals/entities at the selected times using permission schemes and authentication schemes as defined by the individual. The escrow server further provides that the underlying permission/authentication schemes are not generally made available and are executed by the escrow server without revealing or compromising sensitive personal information.

In the present invention a user selecting an action to be performed by the intelligent personal health management appliance/personal visualization appliance/a mobile device or a stationary device connects to a central server/other intelligent personal health management appliances of the present invention by wired or wireless means. The central server receives the request for the action, and parses the necessary information to service the information/action request. Reference to the central server is intended to include one or more other servers such a local server, a network server and an escrow server. Any information may be parsed, but will normally include identification (ID) of the user, specific identification information related to the intelligent personal health management appliance, the servers and other devices; information about the selected input/output formats selected, communication protocols selected, language, the basis or type of the action, the number of actions desired, and any parameters that may be pertinent.

The parameters involved may include, for example, inputting a limit on the values for a comparison, such as a limit on a selected health parameter/a selected visualization parameter by itself or in relation/conjunction with other parameters including gastronomic parameters.

Another preferred embodiment of the present invention includes a mobile device such as a cellular telephone, personal digital assistant, lap top computer and other mobile devices for enabling the control of one or more intelligent personal health management appliances/personal visualization/dental hygiene appliances and servers, for initiating a selected query/action using the user preferred input/output methods, displaying a response to the inquiry from the information server/other intelligent appliances on the display device/mobile device/intelligent personal health management appliance/personal visualization appliance and a personal dental hygiene appliance.

The preferred embodiment includes sending the inquiry from the display device/mobile device to the information server. The preferred embodiment also includes preparing a response to the inquiry on the information server. The mobile device is used for receiving the response to the personal health management inquiry from the information server with the display device/mobile device. The preferred embodiment includes formatting the response to the personal health management/personal visualization inquiry on the display device/mobile device/personal health management appliance/personal visualization appliance/personal dental hygiene appliance, and displaying the formatted response to the personal health management inquiry on the display device/mobile device/intelligent personal health management appliance/personal intelligent visualization appliance/personal intelligent dental hygiene appliance.

The novel embodiments and teachings of the present invention will be made clear in conjunction with the figures and the descriptions:

Referring now to FIG. 1 in detail, the personal intelligent visualization appliance [IVA] 100 comprises of various components and functions represented for convenience of illustration by various functional blocks; such as the mechanism and electronics block 101 comprising of mechanism for providing mechanical motion, ultrasound or other motions, various electronics including a processor[s] and storage; a battery/line power block 102; an optional cleaning fluid reservoir or replaceable cleaning fluid cartridges represented by block 103; a transmitter/receiver and communication block 104 for wired or wireless communication using one or more types of communication protocols via the communication path 140; an external input/output jack represented by block 105; an input/output and display block represented by 110 for enabling different types of input/output and display including touch screen, voice, audio, image, video, data and other formats; a light/other types of imaging sources for enabling optical imaging, infrared imaging, X-ray imaging, magnetic resonance imaging, ultrasound imaging and other types of imaging; a camera, lens, and other imaging means for generally acquiring the image signal for further processing/storage by the internal electronics of the intelligent visualization appliance by itself or in conjunction with a central server. The communication between various internal functional blocks is represented by the bus 106; and an optional disposable/cleanable transparent sleeve 109 is provided for multiple uses or for use by one or more individuals.

The intelligent visualization appliance 100 is enabled for operation in a standalone manner or in conjunction with a mobile device such as cellular telephones, personal digital assistants, laptop computers/stationary device such as personal computers and other devices by wired or wireless communication via communication path 140 using one or more selected communication protocols that are appropriate for each type of device that the IVA 100 communicates with. The visualization printer 160 enables printing of visual information, and an external display 161 provides added viewing capabilities. The IVA 100 is enabled to work in tandem with a local server 162, a central server 180, an escrow server 181 and one or more Internet servers 182, other intelligent personal health management appliances 170, 171 and 172 via wired or wireless communication path 140.

The individual user is represented by 130, and is enabled to use the IVA 100 in conjunction with a cellular telephone [CT] and other mobile devices such as PDAs and camera phones 120. The CT/MD 120 comprises of a keyboard 121, an auxiliary input/output jack 124, a display including a touch screen display 122 and an antenna 123. The CT/MD enables voice and data communication including audio, video and other forms of input/output. The control path 131 is shown to illustrate that the individual 130 can control the CT/MD for one or more functions and also the IVA 100 indirectly via the CT/MD 120.

Optionally, the individual 130 is enabled with radio frequency identification, RF ID Tag 110 that contains user specific information enabling the user to be recognized for one or more automatic functions. The RF ID Tag 110 is enabled for communication seamlessly with IVA 100, other intelligent appliances and servers via communication path 140 using one or more selected communication protocols. Information may be accessed from the RF ID Tag and or written to the RF ID Tag. Additionally, various intelligent appliances/mobile devices including the IVA 100 may be configured with their own RF ID Tag for unique identification and seamless communication.

The information that has been visualized is enabled for viewing on the display of the IVA 100, on an external display 161 and on a specially configured mirror system 150. The intelligent mirror display system 150 enables the direct viewing in a standard mirror and the contemporaneous viewing of the visualization information from one or more internal/external parts of the human anatomy.

### ILLUSTRATIVE EXAMPLE 1

### Intelligent Mirror System Operation and Use

The intelligent mirror system 150 is also enabled for viewing of the posture [full/partial] of a person and capturing/displaying the posture information in real time or at a selected time. The variation of the posture, especially of the mid torso/stomach area may indicate obesity and other health related problems which could be modulated by diet and exercise. Often visualization of the back and other hard to reach parts of the external human anatomy are not possible unless some one views it and describes what was viewed, which is not satisfactory. Using the intelligent external intelligent visualization appliance an individual is enabled to view, store and communicate visual information to the intelligent mirror display or other displays for desired evaluation. As an example, a skin growth on the back can be viewed and recorded at different time periods by the IVA to determine the rate of growth and additionally communicate the visual information to a health care professional for treatment. Similarly, a mobile device such as a cell phone configured with a camera is enabled for visualization of various external anatomical conditions by itself or in the case of hard to reach and manipulate conditions via a specially designed external intelligent visualization appliance 100. The communication of the visualized images to a health care professional coupled with collateral information provides the ability for remote treatment in a more accurate and efficient manner. Additionally archival of information is enabled for subsequent co-relation with other factors within the IVA 100, within the mobile device and or a server for accurate communication of selected visualization and other information to a health care professional.

### EXAMPLE 2

Visualization in conjunction with cleaning and application of medications. In certain cases the visualization by itself is not sufficient except for providing visual information, but does not generally enable application of topical or other medications accurately to the affected external areas of the human anatomy. Consequently, there is a need for an intelligent visualization appliance that combines the visualization process with the capability for cleaning and or application of selected medications. The optional capabilities provided by a cleaning/medication reservoir/cartridges 103 enables visualization, cleaning and the application of medications to the affected areas.

### EXAMPLE 3

Similarly, the examination of the internal parts of the anatomy such as the examination of the ear canal, nostrils and other cavities is difficult if not impossible for an individual to execute and is too personal for others to view and describe. The CT/MD and other devices configured with a camera can not by their form factor accomplish the task of internal cavity examination of the human anatomy. Consequently, there is a need for specific attachments for visualization, that work in conjunction with a mobile device/stationary device; and there is also a need for a new class of intelligent internal visualization appliances that work in a standalone manner or in conjunction with a mobile/stationary device and one or more servers. An intelligent visualization appliance 100 enables safe inspections with the appropriate use of a specially configured for the selected purpose. The periodic visual examination of private cavities and the secure archival of said visual information is expected to be of value in determining adverse conditions that when detected early could point out malignant conditions that can be treated in a timely manner.

### EXAMPLE 4

### Visualization, Cleaning and Application of Medications

The incidence of cervical cancer, colo-rectal cancer and other diseases is treatable if caught early with proper examination. The ability to visualize various cavities such as the vaginal cavity and rectal cavity and to cleanse it periodically is of significant value in saving lives. Safe intelligent visualization appliances, that in addition perform the functions of cleaning and enable application of medications is of value. The intelligent visualization appliance 100 is enabled for viewing, cleaning and application of medications internal of the cavities of a human anatomy in the selected combination of sequence. The visual information is contemporaneously enabled for viewing on the appliance 100 itself, on a mobile device, on an external display or on an intelligent mirror system 150.

### EXAMPLE 5

### Ultrasound, Infra Red, X-Ray and Other Types of Imaging with the Intelligent Visualization Appliance

The visualization is not intended for optical viewing alone but is intended to include other types of imaging including ultra sound and other methods. The processing/storage/software capability needed for various types of optical and other complex visualization methods is enabled in the present invention within the intelligent visualization appliance itself, within the mobile device, within a server or a combination thereof. The IVA 100 configured with an ultrasound imaging head is enabled for ultrasound imaging of the selected part of the human anatomy. Alternately, portable/mobile intelligent ultrasound and other specialized appliances are enabled for the home environment [home/office/institutional health care networks] in a cost effective manner [including the ability to borrow/rent such equipment] for comprehensive personal health management in the future.

The intelligent visualization appliance 100 is enabled for swapping out various imaging and camera heads/attachments that could be sterilized; including the appliance 100, it self in its entirety. Additionally, the imaging head and various parts of the appliance are configured for flexibility and ease of use for the intended visualization application.

Referring now to FIG. 2 in detail, the intelligent dental hygiene appliance [IDHA] 200 comprises of various elements represented for ease of illustration as functional blocks; such as functional block 201 which includes mechanism and electronics [processor, storage and other elements]; a power source that includes line power/battery represented by 203; a communication functional block 204, for wired or wireless communication including a transmitter/receiver; a input/output jack 209; an internal communication bus for communication between various elements/blocks represented by 208; a tooth paste/cleaning agent reservoir/replaceable cartridge represented by 207; an input/output and display block represented by 206; a camera, lens, light source and imaging system functional block represented by 205; and finally the bristles on the intelligent dental hygiene appliance 210. The IDHA 200 communicates by wired or wireless communication means 260, on one or more communication channels using selected communication protocols that are appropriate for the intended device and intended communication. The mechanism in functional block 201 is intended to optionally provide different types of mechanical motion and agitation as desired/selected by the user including high velocity fluid flow of water and or cleaning agents.

The IDHA 200 is intended to optionally be configured with a embedded or attached RF ID Tag capability to uniquely identify the intelligent appliance. Other types of identification including optical bar code, mobile IP identification and other identification, that enable the IDHA to be uniquely identifiable with a specific user is enabled. The individual 240 is enabled for communication, command and control of the IDHA/mobile device CT/MD 220 by communication path 241. The individual is uniquely identifiable for automatic recognition and other utility via an embedded/wearable RFID Tag 250. The RF ID Tag 250 is enabled for communication by the wired/wireless communication path 260 with the IDHA 200, with the CT/MD 220 and with other intelligent appliance and servers. The information contained in the RFID is accessible by one or more authorized devices and the information within the RFID Tag 250 is enabled for real time updating. The CT/MD 220 comprises of a keyboard 221, a display 223, an antenna 222 and communicates on one or more channels by wired or wireless means 260, using one or more communication protocols.

An intelligent mirror and display system 290 comprises of a mirror 291 and a display 292 for contemporaneous viewing of dental images, teeth and cleaning routines that are tailored for each user and for each tooth or general location within the mouth. The upper teeth are represented by 231 and the lower teeth by 230. Each tooth is individually identifiable and may be visualized using the IDHA 200 and specific information related to the teeth/gums and other areas of the mouth may be visualized and recorded.

The IDHA 200, the RF ID Tag 250 and the CT/MD 220 are enabled for communication with each other and also with the external dental printer 270, the external display 271, the local server 272, the intelligent personal health management appliances 280, 281, 282; and the central server 283, the escrow server 284 and the Internet server 285 by wired or wireless communication path 260.

The functioning and use of the intelligent dental hygiene appliance 200 is better understood with the following illustrative examples:

### EXAMPLE 6

### Customized Dental Hygiene and Teeth Cleaning Routines

The user at an initial time point utilizing the IDHA 200 conducts a visual examination of the mouth and each individual tooth from one or more angles using the flexible head of the IDHA 200 and records the information within the appliance itself and or records the information on a central, local or network server or a combination of the above. The visual information is enabled for viewing on the IDHA 200 or on an external display including the intelligent mirror and display system 290. The identification of the individual is manually entered and the IDHA is assigned to a specific individual and programmed with user specific information. In another embodiment, the IDHA is configured with a RFID Tag which is configured and programmed with user specific information enabling recognized communication within the network. The individual in another embodiment is configured externally with a wearable RFID Tag programmed with customer specific information. The RFID Tag of the appliance 200 and the RFID Tag of the user 250 are enabled for seamless communication.

The user having visualized the condition of the teeth and in fact each specific tooth is enabled to program a customized cleaning routine that assigns the selected time needed and the selected pressure and types of motion needed for achieving the selected level of luster and cleaning. The individual is enabled to contemporaneously view the teeth as they are being cleaned or may view the teeth after cleaning or iteratively.

The visualized dental information related to all the teeth or a specific tooth located either on the top level or the bottom level of the mouth is communicated periodically to the dentist for collaborative dental health management. The types of foods masticated/ingested are known to affect the health of the teeth. The gastronomic information and other related information is tracked with the visual information related to teeth for a comprehensive personal health management by the individual him/her self or in conjunction with a dentist. Alternately the information is communicated and archived within a mobile device for visualization while traveling or at the office. Alternately, the IDHA 200 may be configured as an attachment that works in tandem with a CT/MD 220.

### EXAMPLE 7

The IDHA 200 is optionally configurable by original design or attachments to include a tooth paste reservoir/teeth and mouth cleaning fluid reservoir/or a cartridge[s] of tooth paste/cleaning fluids. The selected quantity of tooth paste/cleaning fluid is initially provided or alternately a programmed quantity of tooth paste/cleaning fluid is provided as per user defined and customized routines for achieving the selected dental hygiene results. The IDHA may be configured to provide voice/audio prompts and visual prompts and feed back during dental hygiene procedures.

### EXAMPLE 8

### Boredom with the Same Tooth Paste and or Cleaning Fluid

Tooth paste is often purchased in a large tube of a single flavor that typically lasts for over a month. Similarly, mouth wash and other oral hygiene fluids of a single flavor are sold in large containers that too last for over a month. Consequently, the individual is bored and could use variety as he/she start their day or end their day. In an embodiment of the present invention, the tooth paste/cleaning fluid reservoirs are integral to the IDHA 200 and are rechargeable with new tooth paste/cleaning fluids. In another embodiment of the present invention, the tooth paste/cleaning is available in cartridges that are disposable, such that a cartridge of a new flavor could be used each day or as desired. Alternately, a number of mini cartridges are enabled such that the user can load the cartridge carousel with one or more same or different type tooth pastes/cleaning fluids for selected and varied use to overcome flavor boredom and or use a selected cartridge for medical reasons such as applying an oral ointment to the gums or selected areas of the mouth.

### EXAMPLE 9

### Custom Blending of Tooth Paste and Cleaning Fluids

Additionally, the tooth paste may be dispensed from a central/distributed orifice in the brush head/cleaning nozzle such that the selected tooth paste of a particular customized flavor is dispensed in the quantity desired from one or more mini cartridges where in each cartridge has a different flavor. Similarly, the cleaning fluids are enabled for custom flavoring.

The tooth paste normally foams, however tooth pastes/cleaning fluids that do not foam or do not foam excessively may be utilized for enhanced visibility/viewing while brushing/cleaning.

### EXAMPLE 10

### Music, Video and Audio while Brushing

The IDHA 200 is additionally enabled or command and control such that user preprogrammed music, audio, video, news and other information is played/displayed on an external display or the intelligent mirror system 290 making the chore of dental hygiene pleasant for children and adults. In another embodiment of the present invention, the act of teeth cleaning [not cleaning on schedule] and other dental hygiene procedures is automatically recorded within the appliance 200 itself, within the mobile device and servers such as a PC located in a home network such that alerts and prompts are provided; these being highly relevant for parents who can determine the activity history of dental hygiene of their children.

### EXAMPLE 11

### Different Types of Cleaning Energy

There is a need for applying different types of motion for different type of teeth on the top, at the bottom, in the back, in the front and so on. The mechanical motion such as up and down movement, rotation and other methods often need to be augmented with other forms of energy such as ultrasound. In another embodiment of the present invention, the mechanism/electronics in functional block 201 enable different types of energies and motions to be applied in a selected and user defined manner.

### EXAMPLE 12

### Interchangeable Brush Heads and Automatic Signaling for Brush Head Change

In another embodiment of the present invention, the IDHA 200 is enabled with various specially designed interchangeable brush heads for different types of brushing and cleaning. The interchangeable brush heads provide targeted cleaning specific to a given tooth/teeth/gums. Similarly, in another embodiment of the present invention, the nozzles and other tips used for delivering different types of cleaning fluids, including fluids with pressurized jets are enabled such that targeted cleaning is enabled.

In another embodiment the IDHA 200 is enabled with sensors and electronic signaling capability to indicate when a specific brush head is installed/put to use and the time at which the brush head needs to be swapped/thrown away. Currently, the decision to discard a brush head is erratic and made without proper assessment. The IDHA 200 of the present invention is programmable for a specified duration that the user selects to use the brush head for cleaning. At the end of this duration the user is alerted that the brush head needs to changed/discarded. Similarly, the cleaning agents/cartridges used are indicated for signaling of change/discarding.

Referring now to FIG. 3, shows a table for illustrative purposes comprising visualization and other relevant information. However, the information may be organized in any number of formats including in different database formats for ease of use with the intelligent visualization appliance 100, the intelligent dental hygiene appliance 200, the CT/MD 120/220, other intelligent personal health management appliances, stationary devices and servers. The objective is to archive visualization and other pertinent information acquired by a specific and identifiable intelligent visualization appliance 100/the intelligent dental hygiene appliance 200 such that the information is easily searchable, easily accessible by the user and other authorized health care professionals by wired or wireless communication over a local network or the Internet using different types of intelligent appliances, mobile devices and stationary devices.

The first column of the table shows the date and time at which specific visualization/dental hygiene and other activities are initiated [and also completed]. The second column shows the information related to a specific and uniquely identifiable first intelligent visualization appliance 1. The third column shows the information related to a specific and uniquely identifiable second intelligent visualization appliance 2. The fourth column shows the information related to a specific and uniquely identifiable first intelligent dental hygiene appliance 1. The fifth column shows the personal notes and alerts and other information set up by the user for a selected user defined environment/action. The sixth column shows the information related to the devices/servers where the information is archived including the storage devices and databases of said devices/servers. The seventh column shows different permissions for use of the IVA 100/IDHA 200 and the information contained therein or on other devices/servers/databases. The permissions enable authenticated individuals to access only the information that they have been authorized to access. The permissions and authorizations are enabled for setting up in a dynamic environment and are capable of being changed and viewed via the intelligent appliance and via the mobile device/stationary device. The eighth column shows the notes of the doctor/health care professional including notes and alerts.

### EXAMPLE 13

### Communication of Visualization Information and Personal Actions/Actions in Conjunction with a Health Care Professional

The user [mother] visualizes a selected area of the anatomy, such the ear of a baby utilizing the IVA 100 at a initial time period T1 and archives the visualized information identifying the data for right ear/left ear including the name, date of birth and other information such as height, weight and so on. The information is enabled for archival within the IVA 100, within the CT/MD 120/220, within a local server such as home PC or other servers.

At a second point in time T2, the baby is irritable complaining of ear ache. The mother uses the IVA 100 and examines the right/left ear canal of the baby for inflammation. The visual information acquired is compared by the mother accessing the initial data from its respective device data base and viewing the information on a display device including the IVA 100 itself, a mobile device, a PC or other display devices.

The mother is able to compare the visualization information at T1 against T2 and determine that there is no inflammation and it does not warrant the care of a professional such as a doctor. Alternately, the mother feels that there is an inflammation and communicates the visualization information at T1 and T2 to a doctor/nurse. The doctor/nurse after looking at the visualization information conclude that there is no cause for alarm or alternately conclude that the mother needs to bring the baby in for a checkup at the doctors office.

### EXAMPLE 14

### Use of a Camera Equipped Mobile Device/Stationary Device

The mother notices a skin condition/so called rash on the baby on the arms and torso. The so called rash has grown in size and has certain defined characteristics. The mother is not sure whether this just prickly heat or measles or other more severe ailments. The mother takes an image/video of the affected areas and archives the information within the CT/MD and a PC or the Internet server. The mother communicates the visualized information to the doctor. The doctor requests additional information such as if the baby had been exposed to different allergic agents, played in the yard/woods and is exhibiting elevated body temperature and so on. Based on the visualization and other collateral information, the mother and the doctor decide that either it warrants an immediate visit to the doctors office or the condition is treatable with topical medications.

The organization shown in FIG. 3 is for illustrative purposes and is not to be construed as limiting. Look up tables and databases containing visualization and other information are intended to be organized in a manner that is appropriate for the processing power/storage resident therein and the processing power/storage and other capabilities that are accessible in real time or at a selected time by wired or wireless communication means using one or more channels of communication of an intelligent appliance/mobile device and a stationary device.

Additionally, the table shown is related to a specific individual such as John Doe, with other pertinent information such as a social security number or other identification; and optionally the RFID Tag associated with that individual which enables automatic recognition and action; and including identification of the individual in conjunction with his/her mobile device. The intelligent appliances such as IVA 100 and IDHA 200 are enabled for identification with an RF ID Tag or other means including mobile IP identification for seamless and authenticated communication, accessing of data/information and the updating of information in the databases resident therein or other servers/devices. The information related to the health care providers and their contact information is enabled in the table of FIG. 3; including the Internet websites/servers where the patient may post/upload the visualization and other information for access and evaluation by the authorized health care professional.

Referring now to FIG. 4, the figure shows the architectural scheme for personal health management utilizing various types of visualization information acquired using the IVA 100, IDHA 200, the CT/MD 120 and other intelligent personal health management appliances. The visualization information shown in block representation 401 is used by itself or in conjunction with other measured, observed and monitored values shown in block 402 obtained via other intelligent personal health management appliances including by automatic means. The measured value information 402 may be blood pressure, temperature and so on. The gastronomic and other pertinent information shown in block 403 is useful in determining relationships between visualization information, measured value information and other information for comprehensive tracking, correlation, evaluation, secure archival and personal health management as shown in block 430 by the individual 410 acting alone or in conjunction with health care professionals 420 to who the required and selected information is provided via block 440 with secure access and permission based access.

The present invention therefore enables individual personal health management or health management in collaboration with one or more health care professionals 440.

The foregoing descriptions of specific embodiments of the present invention have been presented for purposes of illustration and description. They are not intended to be exhaustive or to limit the invention to the precise forms disclosed, and it should be understood that many modifications and variations are possible in light of the above teaching. The embodiments were chosen and described in order to best explain the principles of the invention and its practical application, to thereby enable others skilled in the art to best utilize the invention and various embodiments with various modifications as are suited to the particular use contemplated. It is intended that the scope of the invention be defined by the Claims appended hereto and their equivalents.

1-35. (canceled)

36. A visualization system for visualization of the human anatomy, the system comprising a visualization appliance and a central server coupled via network communication means, wherein the visualization appliance performs visualization of an internal anatomical cavity and external anatomical visualization and comprises:
a processor coupled to at least one of control inputs/outputs, optional ports for connection to an external system, an internal storage device, an internal bus that communicates between different components and subsystems, a display, and a power source;
an imager located internal to the visualization appliance and coupled to the processor, wherein the imager acquires images of the human anatomy;
an energy source coupled to the processor, the energy source including one or more of optical, light, electromagnetic, and X-Ray energy for selectively imaging the human anatomy;
a transmitter/receiver coupled to the processor, wherein the transmitter/receiver transfers voice, audio, images, and data to and from external devices that include servers and mobile devices selected from a group consisting of cellular telephones, personal digital assistants (PDAs), personal computers, and external displays;
at least one of sterilized and disposable components; and
identification means that identify the visualization appliance, the identification including at least one of alpha designation, numeric designation, internet protocol (IP) identification, visual identification, optical identification, electronic identification, and radiofrequency (RF) identification.

37. The visualization system of claim 1, wherein the visualization appliance comprises:
at least one reservoir selected from a group consisting of reservoirs for one time use, refillable reservoirs, cartridges for one time use, and replaceable cartridges, wherein the reservoir contains a material that includes one or more of medications, fluids, pastes, semi-solids, solids, powders, aerosols, gases;
means for delivering the materials to an anatomical location at a selected time and in selected quantity for a selected action including one or more of teeth cleaning, teeth brushing, wherein the delivering occurs at least one of prior to the visualization of the selected anatomy, during the visualization, after the visualization.

38. The visualization system of claim 1, comprising:
means for correlating a specific visualization appliance to a specific individual;
means for dedicated use by that individual of his/her assigned intelligent visualization appliance in conjunction with a user-selected identification method that includes the identification means.

39. The visualization system of claim 1, comprising means for automatically recognizing the visualization appliance using an individual identification means carried on an individuals person.

40. The visualization system of claim 1, wherein the visualization appliance comprises means for visual examination of internal cavities of a human body including one or more of mouth, nose, throat, ears, rectum, vagina.

41. The visualization system of claim 1, wherein the visualization appliance comprises user identification means that enables the visualization appliance to be used by more than one individual with appropriate selection of user identification.

42. The visualization system of claim 1, wherein the visualization appliance can be used for a plurality of purposes with appropriate cleaning and sanitizing procedures.

43. The visualization system of claim 1, cleaning and sanitizing procedures include the at least one of sterilized and disposable components.

44. The visualization system of claim 1, wherein the visualization appliance comprises means for visual examination of external regions of a human body including one or more of skin, head, neck, torso, arms, hands, fingers, legs, feet, toes.

45. The visualization system of claim 1, wherein the processor acquires images, processes images, and stores images, wherein the processor controls communication including secure communication of images, voice, and data.

46. The visualization system of claim 1, wherein the visualization appliance is coupled to at least one network including a home network and a network in a medical facility.

47. The visualization system of claim 1, wherein the visualization appliance comprises means for comparing images from different time periods, wherein the comparing is used for determination, evaluation and treatment, wherein the comparing includes comparing using distributed processing resources of at least one network including a home network and a network in a medical facility.

48. The visualization system of claim 1, wherein the visualization appliance is coupled to at least mobile device and transfers data to the at least one mobile device.

49. The visualization system of claim 1, wherein the visualization appliance comprises means for correlating the images and data with physical data selected from a group consisting of physical activity, exercise, sports, and physical condition.

50. The visualization system of claim 1, wherein the visualization appliance comprises means for correlating the images and data with ingestion data, wherein the ingestion data includes information of ingestion of one or more of food, liquid, and medication.

51. The visualization system of claim 1, wherein the visualization appliance comprises:
means for tracking changes in images and data over time and determining changes in physical condition based on the tracking;
means for automatically communicating the changes to a health care professional.

52. The visualization system of claim 1, wherein the visualization appliance comprises:
means for tracking changes in images and data over time and determining changes in physical condition based on the tracking;
means for correlating the changes with physical data selected from a group consisting of physical activity, exercise, sports, and physical condition;
means for correlating the changes with ingestion data, wherein the ingestion data includes information of ingestion of one or more of food, liquid, and medication.

53. The visualization system of claim 1, wherein the visualization appliance comprises an intelligent mirror system coupled to a display, wherein the display displays the images.

54. The visualization system of claim 1, comprising means for personal health management in conjunction with health care providers using remote and real time acquisition of visualization information and measured physiological data from at least one personal health management appliance.

55. The visualization system of claim 1, wherein the servers include an secure escrow server that maintains and provides images to an authorized third party for personal health management in conjunction with user defined access and permissions.
